(19) United States
(12) Patent Application Publication
(10) Pub. No.: US 2005/0177185 A1
(43) Pub. Date: Aug. 11, 2005

(54) Counterwound coil for embolic protection sheath

(75) Inventors: Wayne A. Becker, Elk River, MN (US); Justin E. Plessel, Fridley, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SCIMED LIFE SYSTEMS, INC.

(21) Appl. No.: 10/772,979

(22) Filed: Feb. 5, 2004

(51) Int. Cl. A61M 29/00

(52) U.S. Cl. 606/200

(57) ABSTRACT

An embolic protection sheath putting an elongate shaft proximate at a distal end and a lumen therethrough. A coil assembly including a first coil defining a lumen. The first coil being wound in a first direction and a second coil wound in a second direction. The second coil being disposed around the first coil. The coil assembly can be attached to the distal end of the shaft and the lumen of the shaft can be in fluid communication with the lumen of the first coil.

### FIELD OF THE INVENTION

The present invention pertains generally to the field of embolic protection. More typically, the present invention pertains to sheaths used for delivery or retrieval of embolic protection filters.

### BACKGROUND OF THE INVENTION

Intravascular devices such as embolic protection filters are generally placed within the lumen of a blood vessel or artery to filter embolic debris dislodged during a therapeutic procedure such as percutaneous transluminal coronary angioplasty (PCTA), percutaneous extraction atherectomy or stent delivery. To filter the dislodged embolic debris, an embolic protection filter can be placed distally of the therapeutic device (e.g. an angioplasty or atherectomy catheter) and deployed within the patient's vessel or artery. The filter is often delivered to the target site in a delivery sheath and removed using a retrieval sheath.

### SUMMARY OF THE INVENTION

The present invention relates generally to the field of embolic protection filters. In an exemplary embodiment, a counterwound coil assembly can be incorporated into a delivery or retrieval sheath before the embolic protection filter. In one embodiment, the counterwound coil assembly is disposed at the distal end of a shaft. In an alternate embodiment, the counterwound coil assembly extends substantially to the full length of the delivery or retrieval sheath.

### DESCRIPTION OF DRAWINGS

FIG. 1 is a cross sectional view of a distal end of a sheath including a counterwound coil assembly containing an embolic protection filter.

FIG. 2 is a perspective view of a counterwound coil.

### DETAIL DESCRIPTION OF THE INVENTION

The following description should be read with reference to the drawings, in which like elements in different drawings are numbered in like fashion. Drawings, which are not necessarily to scale, are depict selected embodiments and are not intended to limit the scope of the invention. Although examples of construction, dimensions, and materials and manufacturing processes are illustrated for the various elements, those skilled in the art will recognize that many of the examples provided have suitable alternatives that may be utilized.

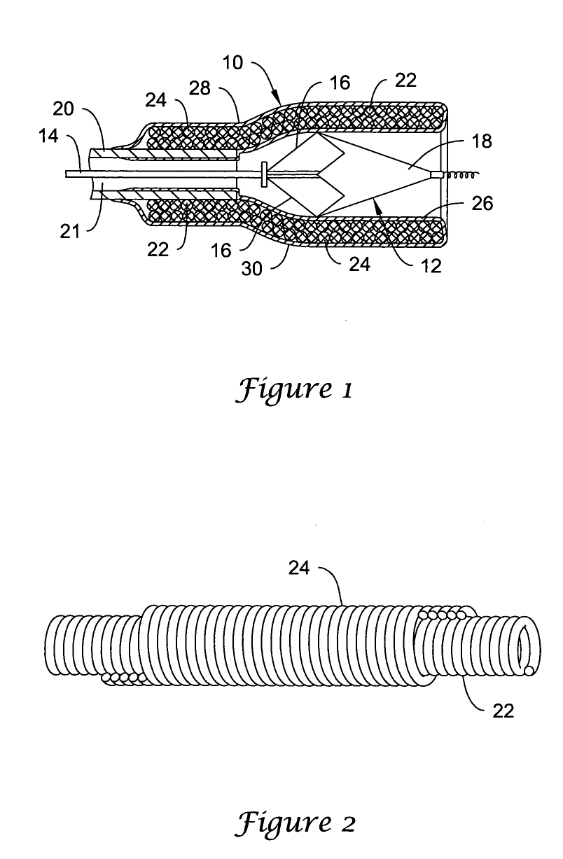

FIG. 1 is a cross sectional view of a distal end of an embolic protection retrieval or delivery sheath 10. Usually the distal end of sheath 10 will have a small ID and OD when configured as a delivery sheath rather than a retrieval sheath. Disposed within delivery sheath 10 is an embolic protection device 12, such as an embolic protection filter. Embolic protection device 12 can be of a type when disposed in sheath 10 is in a compressed position and when disposed outside of sheath 10 is in an expanded position. Embolic protection device 12 can include for example, an elongate shaft 14, such as a guide wire shaft, and plurality of struts 16 to support a filter material 18.

Sheath 10 can include an elongate shaft 20 defining a lumen 21. At the distal end of elongate shaft 20 can be disposed inner coil 22 and outer coil 24. Inner coil 22 and outer coil 24 can be sandwiched between inner tube 26 and outer tube 28. Coils 22 and 24 can be counterwound and multifilar as shown in FIG. 2. The wires used to form coils 22 and 24 can have various cross sections such as circular or rectangular shape. Coils 22 and 24 can taper proximally as shown at 30. Inner and outer tubes 26 and 28 can be a polymer coating or heat shrink polymer bonded to shaft 20. The wires forming inner and outer coils 22 and 24 can also be individually polymer coated.

Shaft 20 can be formed from, for example, a polymeric material such as polypropylene (PP), polyvinylchloride (PVC), polytetrafluoroethylene (PTFE), polyether block amide (PEBA) or other suitable material. Shaft 14, struts 16, filter material 18 and inner and outer coils 22 and 24 respectively may be formed from a metallic material such as stainless steel, platinum, a nickel-titanium alloy or other suitable metal. In the case of filter 18, the metal may be in a form of a wire mesh. Alternately, filter 18 may include a microporous membrane made from a polymeric material such as polypropylene (PP), polyurethane, polyethylene terapthlalate, polyether-ether ketone (PEEK), polyether block amide (PEBA), polyamide (nylon), polyvinylchloride (PVC), polytetrafluoroethylene (PTFE) or any mixture, blend or combination thereof, or other suitable material.

Having thus described the several embodiments to the present invention, those skilled in the art will readily appreciate that other embodiments may be made and used that fall within the scope of claims attached hereto. Numerous advantages of the invention covered by this document have been set forth in the foregoing description. It will be understand that this disclosure is, in many respects, only illustrative. Changes may be made in detail, particularly in matters of shape, size and arrangement of parts without exceeding the scope of the invention.

1. An embolic protection sheath, comprising:
an elongate shaft having a proximal end and a distal end, and a lumen extending threrethrough;
a coil assembly including a first coil defining a lumen, the first coil being wound in a first direction and second coil wound in a second direction, the second coil being disposed around the first coil; and
wherein the coil assembly is attached to the distal end of the shaft, and the lumen of the shaft is fluid communication with the lumen of the first coil.

2. The sheath in accordance with claim 1, wherein the first coil is multifilar.

3. The sheath in accordance with claim 1, wherein the second coil is multifilar.

4. The sheath in accordance with claim 1, wherein the first and second coils are multifilar.

5. The sheath in accordance with claim 1, wherein the first coil includes a wire having a circular cross section.

6. The sheath in accordance with claim 1, wherein the second coil includes a wire having a circular cross section.

7. The sheath in accordance with claim 1, wherein the first and second coils including wires having circular cross sections.

8. The sheath in accordance with claim 1, wherein the first coil includes a wire having a generally rectangular cross section.

9. The sheath in accordance with claim 1, wherein the second coil includes a wire having a generally rectangular cross section.

10. The sheath in accordance with claim 1, wherein the first and second coils include wires having generally rectangular cross sections.

11. The sheath in accordance with claim 1, wherein the coil assembly includes a proximal taper.

12. The sheath in accordance with claim 1, wherein the coil assembly is coated with a polymer.

13. The sheath in accordance with claim 1, wherein the coil assembly is heat bonded to the shaft.

14. The sheath in accordance with claim 1, wherein the first coil includes a polymer coated wire.

15. The sheath in accordance with claim 1, wherein the second coil includes a polymer coated wire.

16. An embolic protection sheath, comprising:
an elongate shaft having a proximal end and a distal end, and a lumen extending threrethrough;
a coil assembly including a first coil defining a lumen, the first coil being wound in a first direction and second coil wound in a second direction, the second coil being disposed around the first coil;
wherein the coil assembly is attached to the distal end of the shaft, and the lumen of the shaft is fluid communication with the lumen of the first coil; and
an embolic protection device including an elongate wire and a filter attached thereto, wherein the wire is disposed at least in part in the shaft lumen.

17. The sheath in accordance with claim 16, wherein the first coil is multifilar.

18. The sheath in accordance with claim 16, wherein the second coil is multifilar.

19. The sheath in accordance with claim 16, wherein the first and second coils are multifilar.

20. The sheath in accordance with claim 16, wherein the first coil includes a wire having a circular cross section.

21. The sheath in accordance with claim 16, wherein the second coil includes a wire having a circular cross section.

22. The sheath in accordance with claim 16, wherein the first and second coils including wires having circular cross sections.

23. The sheath in accordance with claim 16, wherein the first coil includes a wire having a generally rectangular cross section.

24. The sheath in accordance with claim 16, wherein the second coil includes a wire having a generally rectangular cross section.

25. The sheath in accordance with claim 16, wherein the first and second coils include wires having generally rectangular cross sections.

26. The sheath in accordance with claim 16, wherein the coil assembly includes a proximal taper.

27. The sheath in accordance with claim 16, wherein the coil assembly is coated with a polymer.

28. The sheath in accordance with claim 16, wherein the coil assembly is heat bonded to the shaft.

29. The sheath in accordance with claim 16, wherein the first coil includes a polymer coated wire.

30. The sheath in accordance with claim 16, wherein the second coil includes a polymer coated wire.

31. An embolic protection sheath, comprising:
a coil assembly including a first coil defining a lumen, the first coil being wound in a first direction and second coil wound in a second direction, the second coil being disposed around the first coil;
wherein the coil assembly is attached to the distal end of the shaft, and the lumen of the shaft is fluid communication with the lumen of the first coil; and
an embolic protection device including an elongate wire and a filter attached thereto, wherein the wire is disposed at least in part in the shaft lumen.

32. The sheath in accordance with claim 31, wherein the first coil is multifilar.

33. The sheath in accordance with claim 31, wherein the second coil is multifilar.

34. The sheath in accordance with claim 31, wherein the first and second coils are multifilar.

35. The sheath in accordance with claim 31, wherein the first coil includes a wire having a circular cross section.

36. The sheath in accordance with claim 31, wherein the second coil includes a wire having a circular cross section.

37. The sheath in accordance with claim 31, wherein the first and second coils including wires having circular cross sections.

38. The sheath in accordance with claim 31, wherein the first coil includes a wire having a generally rectangular cross section.

39. The sheath in accordance with claim 31, wherein the second coil includes a wire having a generally rectangular cross section.

40. The sheath in accordance with claim 31, wherein the first and second coils include wires having generally rectangular cross sections.

41. The assembly in accordance with claim 31, wherein the coil assembly includes a first diameter section and a second diameter section having a diameter greater than the first diameter section.

42. The sheath in accordance with claim 31, wherein the coil assembly is coated with a polymer.

43. The sheath in accordance with claim 31, wherein the first coil includes a polymer coated wire.

44. The sheath in accordance with claim 31, wherein the second coil includes a polymer coated wire.
